(19) United States
(12) Patent Application Publication
(10) Pub. No.: US 2012/0309034 A1
(43) Pub. Date: Dec. 6, 2012

(54) METHOD FOR MEASURING IMMUNITY OF COMPLEX OF Ku86 AND AUTOANTIBODY THEREOF, KIT USED THEREFOR, AND METHOD FOR DETERMINING CANCER USING SAME

(75) Inventors: Ryo Kojima, Koriyama (JP); Kenta Noda, Koriyama (JP); Masanori Seimiya, Chiba (JP); Kazuyuki Sogawa, Chiba (JP); Fumio Nomura, Chiba (JP)

(73) Assignee: NITTO BOSEKI CO., LTD., Fukushima-shi (JP)

(21) Appl. No.: 13/577,530

(22) PCT Filed: Feb. 7, 2011
(86) PCT No.: PCT/JP2011/052481

§ 371 Date: Aug. 7, 2012

(30) Foreign Application Priority Data
Feb. 12, 2010 (JP) 2010-028387

(51) Int. Cl. G01N 21/59; G01N 33/574; G01N 33/566

(52) U.S. Cl. 435/7.92; 436/501; 435/7.9

(57) ABSTRACT

A reaginic antibody of Ku86 and a substance capable of binding with the autoantibody of Ku86 are acted on a complex of Ku86 and the autoantibody thereof in a subject. Said complex can be measured by measuring the obtained immune complex of the aforementioned complex, reaginic antibody, and substance capable of binding with the autoantibody of Ku86. As a consequence, it is possible to determine whether the cancer is a primary hepatoma, colon cancer, stomach cancer, pancreatic cancer, breast cancer, lung cancer, or an esophageal cancer.

### TECHNICAL FIELD

The present invention relates to a method of immunoassay for measuring a complex between Ku86 and its autoantibody and a kit for the immunoassay. In particular, a complex between Ku86 and its autoantibody occurs specifically in the blood of carcinoma bearing patients at high levels. Thus, the present invention not only provides a method of measuring the complex between Ku86 and its autoantibody, but also may be utilized for determination of carcinoma.

### BACKGROUND ART

Ku86 is a protein which is involved in cleavage of double-stranded DNA, and, together with Ku70, forms a heterodimer, referred to as Ku. That Ku heterodimer has been described as being capable of repairing cleavage of double-stranded DNA in cooperation with a DNA dependent protein kinase and the like (Non Patent Literature 1).

Meanwhile, a modified agarose two-dimensional electrophoresis in which 2D-DIGE technique (two-dimensional fluorescence difference gel electrophoresis) is applied to an agarose two-dimensional electrophoresis (Non Patent Literature 2) has revealed that, by comparing the protein expression levels between a cancerous tissue of primary hepatocellular carcinoma and a peripheral non-cancerous tissue using a proteomic analysis, a protein Ku86 is expressed abundantly in the cancerous area (Non Patent Literature 3).

### CITATION LIST

### Non Patent Literature

Non Patent Literature 1: Li et al., Proc. Natl. Acad. Sci. USA, Vol. 9, No. 2, 832-837, 2002

Non Patent Literature 2: Takeshi Tomonaga et al., Clin. Cancer Res. 2004;10:2007-2014

Non Patent Literature 3: Masanori Seimiya et al., Hepatology 2008;48:519-30

### SUMMARY OF INVENTION

### Technical Problem

The present inventors have found that, by measuring an expression level of Ku86 present in tissue specimens derived from patients suspected of having carcinoma or patients with carcinoma, a cancerous area can be distinguished from a non-cancerous area in those tissues.

The present inventors have further carried on the study on the presence of Ku86 in a blood specimen, and surprisingly found that a complex between Ku86 and its autoantibody may be present in a blood specimen, and the amount of the complex is abundant in the case of patients with carcinoma. Therefore, an object of the present invention is to provide a method of measuring a complex between Ku86 and its autoantibody, which can be applied to the determination of carcinoma.

### Solution to Problem

The present inventors have found that a complex between Ku86 and its autoantibody in a specimen can be measured by immunoassaying the complex, thereby allowing determination of carcinoma, and then completed the present invention.

Thus, the present invention relates to a method of immunoassay for a complex between Ku86 and its autoantibody in a specimen, comprising immunoassaying the complex between Ku86 and its autoantibody in the specimen.

Furthermore, the present invention relates to a kit for immunoassay of a complex between Ku86 and its autoantibody, comprising a reagent antibody against Ku86 and a substance capable of binding to the autoantibody.

Furthermore, the present invention relates to a method of determining carcinoma, wherein the presence of the carcinoma is determined by measuring a complex between Ku86 and its autoantibody.

Furthermore, the present invention relates to a marker for determining carcinoma, comprising a complex between Ku86 and its autoantibody.

### Advantageous Effects of Invention

In the present invention, a complex between Ku86 and its autoantibody present in a specimen, in particular, a specimen derived from blood may be easily measured, which is effective for determination of a patient with carcinoma such as primary hepatocellular carcinoma, colorectal carcinoma, gastric carcinoma, pancreatic carcinoma, breast carcinoma, lung carcinoma, and esophageal carcinoma.

### BRIEF DESCRIPTION OF DRAWINGS

FIG. 1 shows results of measurement of a complex between Ku86 and its autoantibody in serum specimens from healthy individuals, serum specimens from patients with hepatitis C, serum specimens from patients with liver cirrhosis type C, serum specimens from patients with initial primary hepatocellular carcinoma (hereinafter, it may be referred to as initial HCC) and serum specimens from patients with recurrent primary hepatocellular carcinoma (hereinafter, it may be referred to as recurrent HCC), using an ELISA plate sensitized with an anti-Ku86 antibody. In the figure, the horizontal axis represents disease names, and the vertical axis represents absorbance for light with wavelength of 450 nm. An asterisk represents the group of serum specimen in which there are significant difference (p<0.0001) according to Wilcoxon two sample test in all cases when compared to the group of serum specimens from healthy individuals, the group of specimens from patients with hepatitis C or the group of serum specimens from patients with liver cirrhosis type C.

FIG. 2 shows results of measurement of an complex between Ku86 and its autoantibody in serum specimens from healthy individuals, serum specimens from patients with initial HCC, serum specimens from patients with colorectal carcinoma, serum specimens from patients with gastric carcinoma, serum specimens from patients with pancreatic carcinoma, serum specimens from patients with breast carcinoma, serum specimens from patients with lung carcinoma, and serum specimens from patients with esophageal carcinoma, using an ELISA plate sensitized with an anti-Ku86 antibody. In the figure, the horizontal axis represents disease names, and the vertical axis represents absorbance for light with wavelength of 450 nm. An asterisk represents the group of serum specimens in which there are significant difference (p<0.0001) according to Wilcoxon two sample test in all cases when compared to the group of serum specimens from healthy individuals.

### DESCRIPTION OF EMBODIMENTS

Known methods of immunoassay for immunocomplexes between common proteins and their antibodies can be directly applied to the inventive method of immunoassay for a complex between Ku86 and its autoantibody.

In the method of immunoassay of the present invention, a complex between Ku86 and its autoantibody is measured, for example, by reacting the complex in a specimen with a reagent antibody against Ku86 and a substance capable of binding to the autoantibody, and measuring the resulting immunocomplex among the complex, the reagent antibody and the bindable substance.

In the present invention, a specimen is preferably a sample derived from an organism, in particular, a specimen derived from blood is preferred, and examples of a blood specimen include whole blood, blood plasma, and serum.

The subject to be measured by the method of immunoassay of the present invention is a complex between Ku86 and its autoantibody. As described above, Ku86 is a protein which is involved in cleavage of double-stranded DNA, its formal name being ATP-dependent DNA helicase 2 subunit 2. It is otherwise referred to as XRCC5. Additionally, Ku86 is an 82 kDa protein consisting of 732 amino acids, and its accession number (accession No) in US National Center for Biotechnology Information (NCBI) is gi-10863945.

In the present invention, a complex between Ku86 and its autoantibody means an immunocomplex between Ku86 and an autoantibody against Ku86, and may be described herein simply as the complex.

In the present invention, an autoantibody refers to an antibody that is produced by one's own body against substances present in its own body, wherein the substance present in one's own body corresponds to Ku86, and the autoantibody corresponds to an antibody against Ku86.

In the present invention, a reagent antibody against Ku86 refers to an antibody used as a reagent that specifically binds to Ku86, and may be described herein simply as a reagent antibody. That reagent antibody is produced by animal species such as human, mouse, rat, rabbit, goat, and horse, each producing a. given class of immunoglobulin. The reagent antibody may be any of IgG, IgM, IgA, IgE, and IgD. Also, the reagent antibody may be any of a monoclonal antibody, a polyclonal antibody, and fragments thereof (those having an ability to bind to an antigen, for example, H-chain, L-chain, Fab, and F(ab′)2). These reagent antibodies can be obtained as antiserums from the immunized animals generated by immunizing the above described animal species producing antibodies with Ku86 full length proteins or fragment peptides thereof as antigens, or as monoclonal antibodies from the hybridomas obtained by fusing splenocytes from immunized animals and myeloma cells, and screening the fused cells for those producing an antibody against Ku86. As the reagent antibody against Ku86, those products commercially available as an anti-Ku86 antibody may also be used.

In the present invention, a substance capable of binding to the autoantibody is not especially limited as long as it can bind to an autoantibody against Ku86, and may be described herein simply as a bindable substance. As such bindable substances, an anti-IgG antibody, Protein A, Protein G, and a Ku86 antigen as a reagent may be used, and among them an anti-IgG antibody is preferred.

When using a Ku86 antigen as a reagent, though it is not especially limited as long as it is an antigen having antigenic reactivity to an autoantibody against Ku86, examples of it may include a Ku86 full length protein, a variant of the Ku86 full length protein which is a protein retaining the equivalent antigenic reactivity of the full length protein to an autoantibody against Ku86, and having a homology of 90% or more to the amino acid sequence, or a variant protein having an amino acid sequence resulting from deletion, substitution, or addition of one to several amino acid residues in the amino acid sequence of the Ku86 full length protein, and a fragment peptide of Ku86 having antigenic reactivity to an autoantibody against Ku86. Though a Ku86 full length protein is available from Abnova Corporation, the protein or its variant may be synthesized by genetic engineering techniques as its entire amino acid sequence is known. When used in the present invention, a fragment peptide of Ku86 may be generated by cleaving a Ku86 full length protein into various peptide fragments by enzymatic hydrolysis and the like, or may also be easily generated using a commercial automated peptide synthesizer. Additionally, the targeted fragment peptide of Ku86 may be generated by genetic engineering techniques.

The thus-obtained variant or fragment peptide of the Ku86 full length protein may be reacted with an autoantibody against Ku86, then those having antigenic reactivity may be selected to be used as a Ku86 antigen as a reagent. In the present invention, the whole of the each peptide fragment above described, as well as a part of it and the mixture thereof may be used, and are included in a Ku86 antigen as a reagent.

In the present invention, reacting a complex between Ku86 and its autoantibody in a specimen with a reagent antibody against Ku86 and a substance capable of binding to the autoantibody generates an immunocomplex among the complex, the reagent antibody and the bindable substance. To measure the immunocomplex, the immunocomplex may be preferably measured by labeling either of the reagent antibody against Ku86 (the reagent antibody) or the substance capable of binding to the autoantibody (the bindable substance) with a labeling component, and measuring the labeling component in the generated immunocomplex.

As a labeling component, labeling components that are routinely used, such as enzymes, radioactive substances, fluorescent substances, and chemiluminescent substances may be used, but enzymes and radioactive substances are preferred.

As an enzyme used for labeling, an enzyme that is routinely used in enzyme immunoassay (EIA), such as horseradish peroxidase, calf intestine alkaline phosphatase, (β-galactosidase, urease, or glucose oxidase may be used as appropriate, and chromogenic substrates adapted to these enzymes and used routinely in EIA may be used as appropriate. As chromogenic substrates, for example, in the case of HRP, 3, 3′, 5, 5′-tetramethyl benzidine (TMBZ), TMBZ.HCl, TMBZ.PS, ABTS, o-phenylene diamine, p-hydroxyphenyl acetic acid and the like may be used, in the case of alkaline phosphatase, p-nitrophenyl phosphate, 4-methylumbelliferyl phosphate and the like may be used, and in the case of β-galactosidase, o-nitrophenyl-β-D-galactopyranoside, 4-methylumbelliferyl β-D-galactopyranoside and the like may be used.

As examples of radioactive substances used for labeling, for example, a radioactive iodine atom, as fluorescent substances, for example, FITC and rhodamine, and as chemiluminescent substances, for example, luminal may be included.

When using a labeling component in the present invention, for example, the complex is preferably measured by reacting a complex between Ku86 and its autoantibody in a specimen with a reagent antibody against Ku86 bound to a water-insoluble carrier, subsequently reacting the resulting product with a substance capable of binding to the autoantibody, labeled with the labeling component, to generate an immunocomplex among the complex, the reagent antibody, and the bindable substance, and measuring the labeling component bound to the immunocomplex. Alternatively, the complex may be measured by reacting a complex between Ku86 and its autoantibody in a specimen with a substance capable of binding to the autoantibody bound to a water-insoluble carrier, subsequently reacting the resulting product with a reagent antibody against Ku86 labeled with the labeling component, to generate an immunocomplex among the complex, the bindable substance, and the reagent antibody, and measuring the labeling component bound to the immunocomplex.

The preparation of a water-insolubilized carrier may be easily carried out using a known method that binds a protein to the surface of a solid phase. As a solid-phased carrier, for example, beads, microplates, tubes and the like are generally used. For a method of binding a reagent antibody against Ku86 to the surface of these solid-phased carriers, known immobilization techniques such as physisorption and chemical binding may be utilized as appropriate.

Upon contacting the thus solid-phased reagent antibody against Ku86 with a specimen comprising a complex between Ku86 and its autoantibody, the Ku86 portion of the complex binds to the reagent antibody. Further, reacting the bound product with a substance capable of binding to the autoantibody and labeled with a labeling substance (for example, a labeled anti-IgG antibody) generates the immunocomplex among the complex, the reagent antibody and the bindable substance. Consequently, the complex between Ku86 and its autoantibody in the specimen can be measured by measuring the labeling component in the generated immunocomplex.

In a similar way to the above, by binding a substance capable of binding to Ku86 autoantibody to a solid-phased carrier, contacting the solid-phased bindable substance with a specimen comprising a complex between Ku86 and its autoantibody, to allow binding of the autoantibody portion of the complex to the bindable substance, and further reacting the bound product with the reagent antibody against Ku86 and labeled with a labeling substance (for example, an anti-Ku86 antibody) to generate the immunocomplex among the complex, the bindable substance and the reagent antibody, the complex between Ku86 and its autoantibody in the specimen may likewise be measured.

Typical examples of the method of immunoassay of the present invention are shown below.

An anti-Ku86 antibody is added to a plate, the plate is left at rest at low temperature, for example, 4° C. for sensitization, and subsequently washed with a washing solution such as PBS. That plate is then coated with BSA to generate an anti-Ku86 antibody ELISA plate. A diluted specimen is added to the anti-Ku86 antibody ELISA plate, the plate is left at rest at warm temperature, for example at 37° C., and then washed with a washing solution such as PBS. An HRP-labeled anti-Human IgG antibody is added to wells in the resulting plate, the plate is left at rest at warm temperature, for example at 37° C. Then, after washing the wells with a washing solution such as PBS, TMBZ is added thereto, left at rest for example at room temperature, and subsequently 1 N sulfuric acid is added as quencher. For absorbance, absorbance at the wavelength of 450 nm is measured using a microplate reader (from BioRad Laboratories Inc.). The value of a complex between Ku86 and its autoantibody is determined from the absorbance values and the previously made calibration curve.

The method of measuring of the present invention may be performed by a kit for immunoassay of a complex between Ku86 and its autoantibody, comprising a reagent antibody against Ku86 and a substance capable of binding to the autoantibody. The reagent antibody against Ku86 and the substance capable of binding to the autoantibody for this purpose are as described in the method of measuring of the present invention. That is to say, for example, the reagent antibody against Ku86 and the substance capable of binding to the autoantibody, either of which is in a form bound to a water-insoluble carrier, and the other one in a form labeled with a labeling component, may be reagent components of the kit. As additional reagent components, those used routinely in immunoassay such as surfactants, and buffers may be added as appropriate.

In the present invention, carcinoma can be determined by measuring a complex between Ku86 and its autoantibody.

Generally, large amounts of a complex between Ku86 and its autoantibody are suspected as indicating carcinoma such as primary hepatocellular carcinoma (such as primary hepatocellular carcinoma, primary cholangiocellular carcinoma), liver carcinoma such as metastatic liver carcinoma, bladder carcinoma, breast carcinoma, lung carcinoma, ovarian carcinoma, prostate carcinoma, thyroid carcinoma, and skin carcinoma, therefore, it is effective for distinguishing cancerous diseases in patients to measure the autoantibody against Ku86 by the method of measuring of the present invention. Utilizing the method of measuring of the present invention is effective in particular for distinguishing carcinomas selected from the group consisting primary hepatocellular carcinoma, colorectal carcinoma, gastric carcinoma, pancreatic carcinoma, breast carcinoma, lung carcinoma and esophageal carcinoma. For example, it is effective for distinguishing healthy individuals from patients with initial primary hepatocellular carcinoma, patients with recurrent primary hepatocellular carcinoma, patients with colorectal carcinoma, patients with gastric carcinoma, patients with pancreatic carcinoma, patients with breast carcinoma, patients with lung carcinoma or patients with esophageal carcinoma. Also, by utilizing the method of measuring of the present invention, it is possible to distinguish a patient with primary hepatocellular carcinoma such as a patient with new-onset primary hepatocellular carcinoma or a patient with recurrent primary hepatocellular carcinoma from a patient with hepatitis C or a patient with liver disease such as liver cirrhosis type C.

As apparent from the description above, a complex between Ku86 and its autoantibody may be used as a marker for determination of carcinoma, for example, primary hepatocellular carcinoma (such as primary hepatocellular carcinoma, primary cholangiocellular carcinoma), liver carcinoma such as metastatic liver carcinoma, bladder carcinoma, breast carcinoma, lung carcinoma, ovarian carcinoma, prostate carcinoma, thyroid carcinoma, and skin carcinoma. A complex between Ku86 and its autoantibody is also preferred as a marker for determining carcinoma using a specimen derived from blood such as whole blood, blood plasma, serum.

### EXAMPLES

Hereinafter, the present invention will be described in more detail with reference to the examples, but the present invention is not to be limited to these examples in any way.

### Example 1

### Measurement of a Complex Between Ku86 and its Autoantibody

For serum specimens collected from healthy individuals, patients with hepatitis C, patients with liver cirrhosis type C, patients with initial primary hepatocellular carcinoma, patients with recurrent primary hepatocellular carcinoma, patients with colorectal carcinoma, patients with gastric carcinoma, patients with pancreatic carcinoma, patients with breast carcinoma, patients with lung carcinoma, and patients with esophageal carcinoma, a complex between Ku86 and its autoantibody was measured in the manner described specifically below.

### 1. Method

### (1) Generation of an Anti-Ku86 Antibody ELISA Plate

An ELISA plate (from Nunc International, Maxisorp) was sensitized by leaving at rest an anti-XRCC5 antibody(from Abnova Corporation, 5 μg/ml, 100 μL/well)as an anti-Ku86 antibody on the plate overnight at 4° C., and then washed with PBS containing 0.05% Tween20 (200 μL/well) three times. The plate was then coated overnight with PBS containing 1.5% BSA, 10% saccharose (200 μL/well) to generate the anti-Ku86 antibody ELISA plate.

### (2) Measurement of the Complex Between Ku86 and Its Autoantibody

An HRP-labeled anti-Human IgG antibody (from Zymed Laboratories Inc.) as a detection antibody diluted 4000 times with PBS containing 0.05% Tween20 was used. Sample serum was diluted 100 times with PBS. The diluted sample was added to the anti-Ku86 antibody ELISA plate in an amount of 100 μL/well, the plate was left at rest for 1 hour at 37° C., and then washed with PBS containing 0.05% Tween20 (200 μL/well) 3 times. To each well of the resulting plate, 100 μL/well of a diluted HRP-labeled anti-Human IgG antibody was added, the plate was left at rest for 30 min at 37° C. Then, after washing the plate with PBS containing 0.05% Tween20 (200 μL/well) three times, TMBZ was added to the plate in an amount of 100 μL/well. After leaving the plate at rest for 10 minutes at room temperature, 100 μL/well of 1 N sulfuric acid as a quencher was added to the plate. Absorbance was measured using a microplate reader (from BioRad Laboratories Inc.) at the wavelength of 450 nm.

It is noted that specimens from 48 healthy individuals, 19 hepatitis C patients, 18 liver cirrhosis type C patients, 32 specimens from new-onset primary hepatocellular carcinoma patients, 27 recurrent primary hepatocellular carcinoma patients, 16 specimens from colorectal carcinoma patients, 16 gastric carcinoma patients, 16 pancreatic carcinoma patients, 16 breast carcinoma patients, 16 lung carcinoma patients, 16 esophageal carcinoma patients were used.

### 2. Results

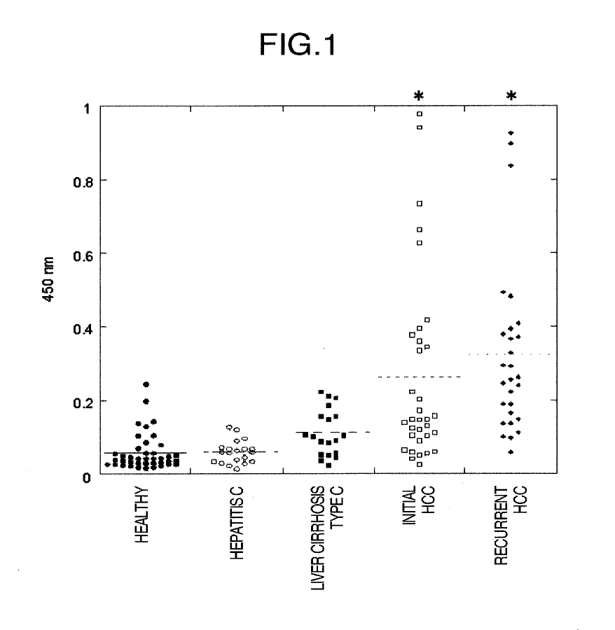

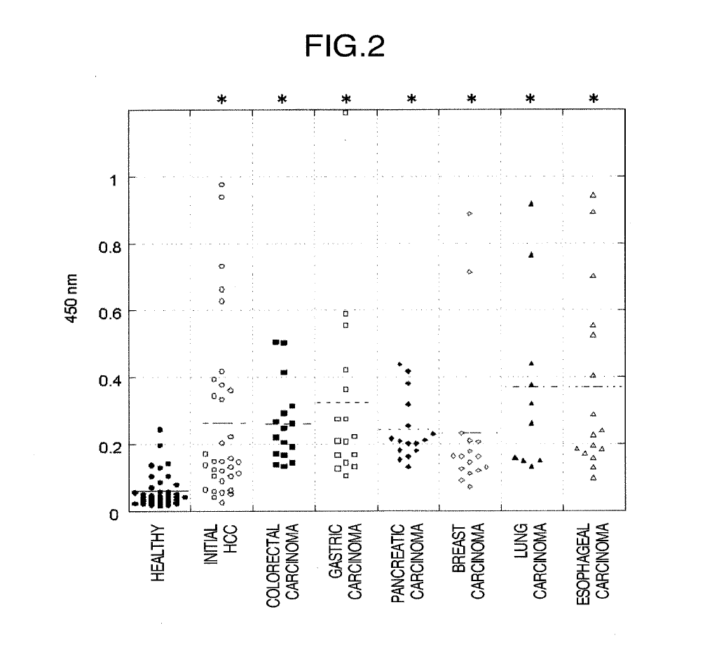

Results of the measurement of a complex between Ku86 and its autoantibody were shown in FIG. 1 and FIG. 2. For significant difference test, KaleidaGraph 4.0 was used, and statistical processing was carried out with Wilcoxon two sample test.

As shown in FIG. 1, as compared to the group of healthy individuals, the group of hepatitis C and the group of liver cirrhosis type C, obvious significant differences in the amounts of the complex between Ku86 and its autoantibody were observed in the group of specimen from patients with initial primary hepatocellular carcinoma and the group of specimen from patients with recurrent primary hepatocellular carcinoma. Therefore, immunoassay of the complex between Ku86 and its autoantibody in blood was shown to be particularly effective for distinguishing primary hepatocellular carcinoma among liver diseases.

As shown in FIG. 2, as compared to the group of healthy individual, obvious significant differences in the amounts of the complex between Ku86 and its autoantibody were observed in the group of specimens from patients with various carcinoma, i.e., the group of specimens from patients with initial primary hepatocellular carcinoma, the group of specimens from patients with colorectal carcinoma, the group of specimens from patients with gastric carcinoma, the group of specimens from patients with pancreatic carcinoma, the group of specimens from patients with breast carcinoma, the group of specimens from patients with lung carcinoma and the group of specimens from patients with esophageal carcinoma. Therefore, immunoassay of the complex between Ku86 and its autoantibody in blood was shown to be effective for distinguishing a patient with carcinoma from healthy individuals, in various carcinomas.

### INDUSTRIAL APPLICABILITY

As described in detail hereinbefore, a complex between Ku86 and its autoantibody can be measured by reacting the complex in a specimen such as a specimen derived from blood with a reagent antibody against Ku86 and a substance capable of binding to the autoantibody, and measuring the resulting immunocomplex among the complex, the reagent antibody and the bindable substance, thereby allowing determination of carcinoma such as primary hepatocellular carcinoma, colorectal carcinoma, gastric carcinoma, pancreatic carcinoma, breast carcinoma, lung carcinoma and esophageal carcinoma.

1. A method of immunoassay for a complex between Ku86 and its autoantibody in a specimen, comprising immunoassaying the complex between Ku86 and its autoantibody in the specimen.

2. The method of immunoassay according to claim 1, wherein the complex between Ku86 and its autoantibody is measured by reacting the complex in a specimen with a reagent antibody against Ku86 and a substance capable of binding to the autoantibody, and measuring the resulting immunocomplex among the complex, the reagent antibody and the bindable substance.

3. The method of immunoassay according to claim 2, wherein either the reagent antibody or the bindable substance is labeled with a labeling component, and the complex is measured by measuring the labeling component in the immunocomplex to measure the immunocomplex.

4. The method of immunoassay according to claim 3, wherein the complex between Ku86 and its autoantibody is measured by reacting the complex in a specimen with the reagent antibody against Ku86 bound to a water-insoluble carrier, subsequently reacting the carrier-bound product with the substance capable of binding to the autoantibody and labeled with the labeling component, to generate the immunocomplex among the complex, the reagent antibody, and the bindable substance, and measuring the labeling component bound to the immunocomplex.

5. The method of immunoassay according to claim 2, wherein the substance capable of binding to the autoantibody is an anti-IgG antibody.

6. The method of immunoassay according to claim 3, wherein the labeling component is an enzyme or a radioactive substance.

7. The method of immunoassay according to claim 1, for determination of carcinoma.

8. The method of immunoassay according to claim 7, wherein the carcinoma is primary hepatocellular carcinoma.

9. The method of immunoassay according to claim 1, wherein the specimen is a specimen derived from blood.

10. A kit for immunoassay of a complex between Ku86 and its autoantibody, comprising a reagent antibody against Ku86 and a substance capable of binding to Ku86 autoantibody.

11. A method of determining carcinoma, wherein the carcinoma is determined to be present by measuring a complex between Ku86 and its autoantibody.

12. The method of determining carcinoma according to claim 11, wherein the complex in a specimen derived from blood is measured.

13. The method of determining carcinoma according to claim 12, wherein the carcinoma is selected from the group consisting of primary hepatocellular carcinoma, colorectal carcinoma, gastric carcinoma, pancreatic carcinoma, breast carcinoma, lung carcinoma and esophageal carcinoma.

14. A marker for determining carcinoma, comprising a complex between Ku86 and its autoantibody.

15. The marker for determining carcinoma according to claim 14, for determining the carcinoma using a specimen derived from blood.

16. The marker for determining carcinoma according to claim 14, wherein the carcinoma is selected from the group consisting of primary hepatocellular carcinoma, colorectal carcinoma, gastric carcinoma, pancreatic carcinoma, breast carcinoma, lung carcinoma and esophageal carcinoma.
